(19) United States
(12) Patent Application Publication
(10) Pub. No.: US 2026/0208155 A1
(43) Pub. Date: Jul. 23, 2026

(54) METHOD FOR PRODUCING SUPERABSORBENTS

(71) Applicant: BASF SE, Ludwigshafen Am Rhein (DE)

(72) Inventors: Monte Alan PETERSON, Freeport, TX (US); Vernon Lynn ADEY, Freeport, TX (US); Jan Niclas GORGES, Ludwigshafen am Rhein (DE); Juergen SCHROEDER, Ludwigshafen am Rhein (DE)

(73) Assignee: BASF SE, Ludwigshafen Am Rhein (DE)

(21) Appl. No.: 19/140,478

(22) PCT Filed: Dec. 12, 2023
(86) PCT No.: PCT/EP2023/085280

§ 371 Date: Jun. 18, 2025

(30) Foreign Application Priority Data
Dec. 20, 2022 (EP) 22214813.2

(51) Int. Cl. B01J 20/26; A61L 15/24; A61L 15/60; B01D 53/14; B01D 53/18; B01J 20/28; B01J 20/30

(52) U.S. Cl. CPC B01J 20/267; A61L 15/24; A61L 15/60; B01D 53/1487; B01D 53/18; B01J 20/28047; B01J 20/3021; B01J 20/3078; B01D 2252/10; B01D 2257/708

(57) ABSTRACT

The present invention relates to a process for continuously producing superabsorbents, where an aqueous monomer solution is polymerized to a polymer gel, the acrylic acid present in the offgas from the polymerization and/or drying is scrubbed out by means of an aqueous solution, the pH of the aqueous solution is from 9.0 to 12.5 and the acrylic acid-laden aqueous solution is used at least partly for production of the monomer solution or metered into the polymerization in parallel with the monomer solution.

The present invention relates to a process for continuously producing superabsorbents, where an aqueous monomer solution is polymerized to a polymer gel, the acrylic acid present in the offgas from the polymerization and/or drying is scrubbed out by means of an aqueous solution, the pH of the aqueous solution is from 9.0 to 12.5 and the acrylic acid-laden aqueous solution is used at least partly for production of the monomer solution or metered into the polymerization in parallel with the monomer solution.

Superabsorbents are used to produce diapers, tampons, sanitary napkins and other hygiene articles, but also as water-retaining agents in market gardening. Superabsorbents are also referred to as water-absorbing polymers.

The production of superabsorbents is described in the monograph “Modern Superabsorbent Polymer Technology”, F. L. Buchholz and A. T. Graham, Wiley-VCH, 1998, pages 71 to 103.

EP 0 922 717 A1 discloses a process for producing superabsorbents by static polymerization. This is done using an inert gas stream for cooling. The inert gas stream can be recycled after condensation of water and acrylic acid. The dilute aqueous acrylic acid obtained by condensation can be used for production of the monomer solution.

WO 2010/040465 A1 discloses the alkaline scrubbing of offgases obtained in the production of superabsorbents, and the recycling of the scrubber water into the process.

EP 1 178 059 A2, WO 2003/051415 A1, WO 2010/040466 A1 and WO 2011/120746 A1 likewise mention the recycling of acrylic acid.

It was an object of the present invention to provide an improved process for producing superabsorbents, especially improved recycling of the acrylic acid discharged from the polymerization reactor with the offgas.

The object was achieved by a process for continuously producing superabsorbents, by polymerizing an aqueous monomer solution comprising a partly neutralized acrylic acid to a polymer gel, optionally extruding the polymer gel, drying the polymer gel and comminuting, classifying and optionally thermally surface postcrosslinking the dried polymer gel, wherein the water content of the aqueous monomer solution is from 40% to 75% by weight, the acrylic acid has been neutralized to an extent of 40 to 85 mol %, the acrylic acid present in the offgas from the polymerization and/or drying is scrubbed out by means of an aqueous solution, the pH of the aqueous solution is from 9.0 to 12.5 and the acrylic acid-laden aqueous solution is used at least partly for production of the monomer solution or metered into the polymerization in parallel with the monomer solution.

The pH of the aqueous solution is preferably from 9.5 to 12.0, more preferably from 10.0 to 11.5, most preferably from 10.5 to 11.0. The pH can be adjusted by mixing in a neutralizing agent. Suitable neutralizing agents are, for example, alkali metal hydroxides, alkali metal oxides, alkali metal carbonates or alkali metal hydrogencarbonates, and mixtures thereof. Particularly preferred alkali metals are sodium and potassium, but very particular preference is given to sodium hydroxide, sodium carbonate or sodium hydrogencarbonate and also mixtures thereof, especially sodium hydroxide. For an acidic pH, correspondingly less neutralizing agent is used. The desired acidic pH is then established automatically by the acrylic acid that has been scrubbed out.

The present invention is based on the finding that the pH of the aqueous solution when the acrylic acid is scrubbed out has an effect on the stability and color of the acrylic acid-laden aqueous solutions obtained.

The aqueous solution is preferably at a temperature of 40 to 80° C., more preferably of 45 to 75° C., most preferably of 50 to 70° C. The amount of water that condenses out too can be influenced via the temperature.

In a preferred embodiment of the present invention, the acrylic acid is scrubbed out of the offgas by means of a scrubbing column. The scrubbing column may have the customary internals. Random packings are preferred.

The gas velocity in the scrubbing column is preferably from 0.2 to 3.0 m/s, more preferably from 0.5 to 2.5 m/s, most preferably from 1.0 to 2.0 m/s. The liquid hourly space velocity in the scrubbing column is preferably from 2 to 50 m³/h, more preferably from 5 to 40 m³/h, most preferably from 10 to 30 m³/h, in each case per m² of internal cross-sectional area of the scrubbing column.

The aqueous solution in the scrubbing column may be partly circulated, preferably from 95% to 99.9%, more preferably from 96% to 99.8%, most preferably from 97% to 99.7%.

The acrylic acid-laden aqueous solution should preferably comprise only less than 99% by weight, more preferably less than 98% by weight, most preferably less than 97% by weight, of water.

The content of acrylic acid and of neutralized acrylic acid in the aqueous solution can be determined by online analysis. The concentrations in the monomer solution can thus easily be kept constant in spite of recycling.

NIR spectroscopy, for example, is suitable for online analysis. The concentration of the components can be determined directly using suitable calibration curves. The concentration of the components can also be determined indirectly via pH, density and temperature. If the pH of the aqueous solution and the temperature are kept sufficiently constant, the concentration of the components can also be determined solely via the density.

The acrylic acid-laden aqueous solution used for production of the monomer solution can lead to a distinct change in centrifuge retention capacity (CRC) and in the extractables content. By online analysis and adjustment of the amounts of acrylic acid, water and/or neutralizing agent feedstocks, it is easily possible to keep the concentrations in the monomer solution constant in spite of recycling.

The production of the superabsorbents is described in detail hereinafter:

The superabsorbents are produced by polymerizing a monomer solution and are typically water-insoluble.

The ethylenically unsaturated monomers bearing acid groups are preferably water-soluble, i.e. their solubility in water at 23° C. is typically at least 1 g/100 g of water, preferably at least 5 g/100 g of water, more preferably at least 25 g/100 g of water and most preferably at least 35 g/100 g of water.

Suitable monomers are, for example, ethylenically unsaturated carboxylic acids, such as acrylic acid, methacrylic acid and itaconic acid. Particularly preferred monomers are acrylic acid and methacrylic acid. Very particular preference is given to acrylic acid.

The ethylenically unsaturated monomers bearing acid groups have typically been partly neutralized. The neutralization is conducted at the monomer stage. This is typically accomplished by mixing in the neutralizing agent as an aqueous solution or else preferably as a solid. The degree of neutralization is preferably from 40 to 85 mol %, more preferably from 50 to 80 mol % and most preferably from 60 to 75 mol %, for which the customary neutralizing agents can be used, preferably alkali metal hydroxides, alkali metal oxides, alkali metal carbonates or alkali metal hydrogencarbonates and also mixtures thereof. Instead of alkali metal salts, it is also possible to use ammonium salts. Particularly preferred alkali metals are sodium and potassium, but very particular preference is given to sodium hydroxide, sodium carbonate or sodium hydrogencarbonate and also mixtures thereof, especially sodium hydroxide.

The monomers typically comprise polymerization inhibitors, preferably hydroquinone monoethers, as storage stabilizers.

Suitable crosslinkers are compounds having at least two groups suitable for crosslinking. Such groups are, for example, ethylenically unsaturated groups which can be polymerized free-radically into the polymer chain, and functional groups which can form covalent bonds with the acid groups of the monomer. In addition, polyvalent metal salts which can form coordinate bonds with at least two acid groups of the monomer are also suitable as crosslinkers.

Suitable crosslinkers are, for example, ethylene glycol dimethacrylate, diethylene glycol diacrylate, polyethylene glycol diacrylate, allyl methacrylate, trimethylolpropane triacrylate, triallylamine, tetraallylammonium chloride, tetraallyloxyethane, as described in EP 0 530 438 A1, di- and triacrylates, as described in EP 0 547 847 A1, EP 0 559 476 A1, EP 0 632 068 A1, WO 93/21237 A1, WO 03/104299 A1, WO 03/104300 A1, WO 03/104301 A1 and DE 103 31 450 A1, mixed acrylates which, as well as acrylate groups, comprise further ethylenically unsaturated groups, as described in DE 103 31 456 A1 and DE 103 55 401 A1, or crosslinker mixtures, as described, for example, in DE 195 43 368 A1, DE 196 46 484 A1, WO 90/15830 A1 and WO 02/032962 A2.

The amount of crosslinker is preferably 0.05% to 1.5% by weight, more preferably 0.1% to 1% by weight and most preferably 0.15% to 0.6% by weight, calculated in each case on the basis of the total amount of monomer used. With rising crosslinker content, the centrifuge retention capacity (CRC) falls and the absorption under a pressure of 21.0 g/cm² (AUL0.3 psi) passes through a maximum.

Initiators used may be all compounds which generate free radicals under the polymerization conditions, for example thermal initiators, redox initiators or photoinitiators. Suitable redox initiators are sodium peroxodisulfate/ascorbic acid, hydrogen peroxide/ascorbic acid, sodium peroxodisulfate/sodium bisulfite and hydrogen peroxide/sodium bisulfite. Preference is given to using mixtures of thermal initiators and redox initiators, such as sodium peroxodisulfate/hydrogen peroxide/ascorbic acid. The reducing component used is preferably the disodium salt of 2-hydroxy-2-sulfonatoacetic acid or a mixture of the sodium salt of 2-hydroxy-2-sulfinatoacetic acid, the disodium salt of 2-hydroxy-2-sulfonatoacetic acid and sodium bisulfite. Such mixtures are obtainable as Brüggolite® FF6 and Brüggolite® FF7 (Brüggemann Chemicals; Heilbronn; Germany).

The water content of the monomer solution is preferably from 40% to 75% by weight, more preferably from 45% to 70% by weight and most preferably from 50% to 65% by weight. As the water content rises, the energy expenditure in the subsequent drying rises and, as the water content falls, the heat of polymerization can only be removed inadequately.

The temperature of the monomer solution is preferably from 10 to 90° C., particularly preferably from 20 to 70° C., very particularly preferably from 30 to 50° C.

For optimal action, the preferred polymerization inhibitors require dissolved oxygen. The monomer solution can therefore be freed of dissolved oxygen before the polymerization by inertization, i.e. flowing an inert gas through, preferably nitrogen or carbon dioxide. The oxygen content of the monomer solution is preferably lowered before the polymerization to less than 1 ppm by weight, more preferably to less than 0.5 ppm by weight, most preferably to less than 0.1 ppm by weight.

Suitable reactors for the polymerization are, for example, kneading reactors or belt reactors. In the kneader, the polymer gel formed in the polymerization of an aqueous monomer solution or suspension is comminuted continuously by, for example, contrarotatory stirrer shafts, as described in WO 2001/038402 A1. Polymerization on the belt is described, for example, in DE 38 25 366 A1 and U.S. Pat. No. 6,241,928. Polymerization in a belt reactor forms a polymer gel which has to be comminuted, for example in an extruder or kneader.

To improve the drying properties, the comminuted polymer gel obtained by means of a kneader can additionally be extruded.

The polymer gel is then typically dried with an air circulation belt drier until the residual moisture content is preferably 0.5% to 10% by weight, more preferably 1% to 7% by weight and most preferably 2% to 5% by weight, the residual moisture content being determined by EDANA recommended test method No. WSP 230.2-05 “Mass Loss Upon Heating”. In the case of too high a residual moisture content, the dried polymer gel has too low a glass transition temperature T_{g} and can be processed further only with difficulty. In the case of too low a residual moisture content, the dried polymer gel is too brittle and, in the 20 subsequent comminution steps, undesirably large amounts of polymer particles with an excessively low particle size are obtained (“fines”). The solids content of the polymer gel before the drying is preferably from 25% to 90% by weight, more preferably from 35% to 70% by weight, most preferably from 40% to 60% by weight. Subsequently, the dried polymer gel is crushed and optionally coarsely comminuted.

Thereafter, the dried polymer gel is typically ground and classified, and the apparatus used for grinding may typically be single- or multistage roll mills, preferably two- or three-stage roll mills, pin mills, hammer mills or vibratory mills.

The average particle size of the polymer particles removed as the product fraction is preferably from 150 to 850 μm, more preferably from 250 to 600 μm, very particularly from 300 to 500 μm. The average particle size of the product fraction may be determined by means of EDANA recommended test method No. WSP 220.2 (05) “Particle Size Distribution”, where the proportions by mass of the screen fractions are plotted in cumulative form and the average particle size is determined graphically. The average particle size here is the value of the mesh size which arises for a cumulative 50% by weight.

To further improve the properties, the polymer particles can be thermally surface postcrosslinked. Suitable surface postcrosslinkers are compounds which comprise groups which can form covalent bonds with at least two carboxylate groups of the polymer particles. Suitable compounds are, for example, polyfunctional amines, polyfunctional amido amines, polyfunctional epoxides, as described in EP 0 083 022 A2, EP 0 543 303 A1 and EP 0 937 736 A2, di- or polyfunctional alcohols, as described in DE 33 14 019 A1, DE 35 23 617 A1 and EP 0 450 922 A2, or β-hydroxyalkylamides, as described in DE 102 04 938 A1 and U.S. Pat. No. 6,239,230.

The amount of surface postcrosslinker is preferably 0.001% to 2% by weight, more preferably 0.01% to 1% by weight and most preferably 0.03% to 0.7% by weight, based in each case on the polymer particles.

In a preferred embodiment of the present invention, polyvalent cations are applied to the particle surface in addition to the surface postcrosslinkers.

The polyvalent cations usable in the process of the invention are, for example, divalent cations such as the cations of zinc, magnesium, calcium and strontium, trivalent cations such as the cations of aluminum, iron, chromium, rare earths and manganese, tetravalent cations such as the cations of titanium and zirconium. Possible counterions are chloride, bromide, hydroxide, sulfate, hydrogensulfate, carbonate, hydrogencarbonate, nitrate, phosphate, hydrogenphosphate, dihydrogenphosphate and carboxylate, such as acetate and lactate. Aluminum hydroxide, aluminum sulfate and aluminum lactate are preferred.

The amount of polyvalent cation used is, for example, 0.001% to 1.5% by weight, preferably 0.005% to 1% by weight and more preferably 0.02% to 0.8% by weight, based in each case on the polymer.

The surface postcrosslinking is typically performed in such a way that a solution of the surface postcrosslinker is sprayed onto the dried polymer particles. After the spray application, the surface postcrosslinker-coated polymer particles are subjected to thermal treatment.

The spray application of a solution of the surface postcrosslinker is preferably performed in mixers with moving mixing tools, such as screw mixers, disk mixers and paddle mixers. Particular preference is given to horizontal mixers such as paddle mixers, very particular preference to vertical mixers. The distinction between horizontal mixers and vertical mixers is made by the position of the mixing shaft, i.e. horizontal mixers have a horizontally mounted mixing shaft and vertical mixers have a vertically mounted mixing shaft. Suitable mixers are, for example, horizontal Pflugschar® plowshare mixers (Gebr. Lödige Maschinenbau GmbH; Paderborn; Germany), Vrieco-Nauta continuous mixers (Hosokawa Micron BV; Doetinchem; the Netherlands), Processall Mixmill mixers (Processal Incorporated; Cincinnati; USA) and Schugi Flexomix® (Hosokawa Micron BV; Doetinchem; the Netherlands). However, it is also possible to spray on the surface postcrosslinker solution in a fluidized bed.

The surface postcrosslinkers are typically used in the form of an aqueous solution. The penetration depth of the surface postcrosslinker into the polymer particles can be adjusted via the content of nonaqueous solvent and total amount of solvent.

The thermal treatment is preferably conducted in contact driers, more preferably paddle driers, most preferably disk driers. Suitable driers are, for example, Hosokawa Bepex® Horizontal Paddle Dryer (Hosokawa Micron GmbH; Leingarten; Germany), Hosokawa Bepex® Disk Dryer (Hosokawa Micron GmbH; Leingarten; Germany), Holo-Flite® driers (Metso Minerals Industries Inc.; Danville; USA) and Nara Paddle Dryer (NARA Machinery Europe; Frechen; Germany). Moreover, fluidized bed driers may also be used.

The surface postcrosslinking can be effected in the mixer itself, by heating the jacket or blowing in warm air. Equally suitable is a downstream drier, for example a tray drier, a rotary tube oven or a heatable screw. It is particularly advantageous to effect mixing and thermal surface postcrosslinking in a fluidized bed drier.

Preferred reaction temperatures are in the range of 100 to 250° C., preferably 110 to 220° C., more preferably 120 to 210° C., most preferably 130 to 200° C. The preferred dwell time at this temperature is preferably at least 10 minutes, more preferably at least 20 minutes, most preferably at least 30 minutes, and typically at most 60 minutes.

Subsequently, the surface postcrosslinked polymer particles can be classified again, with excessively small and/or excessively large polymer particles being removed and recycled into the process.

To further improve the properties, the surface postcrosslinked polymer particles can be coated or remoisturized.

The remoisturizing is preferably performed at 30 to 80° C., more preferably at 35 to 70° C., most preferably at 40 to 60° C. At excessively low temperatures the polymer particles tend to form lumps, and at higher temperatures water already evaporates to a noticeable degree. The amount of water used for remoisturizing is preferably from 1% to 10% by weight, more preferably from 2% to 8% by weight and most preferably from 3% to 5% by weight. The remoisturizing increases the mechanical stability of the polymer particles and reduces their tendency to static charging. The remoisturizing is advantageously performed in a cooler after the thermal surface postcrosslinking.

Suitable coatings for improving the swell rate and the gel bed permeability (GBP) are, for example, inorganic inert substances, such as water-insoluble metal salts, organic polymers, cationic polymers and di- or polyvalent metal cations. Suitable coatings for dust binding are, for example, polyols. Suitable coatings for counteracting the undesired caking tendency of the polymer particles are, for example, fumed silica, such as Aerosil® 200, precipitated silica, such as Sipernat® D17, and surfactants, such as Span® 20.

### EXAMPLE

By continuously mixing deionized water, 50% by weight sodium hydroxide solution and acrylic acid, a monomer solution is prepared such that the degree of neutralization corresponds to 72.0 mol %. The water content of the monomer solution is 57.5% by weight.

The crosslinker used is 3-tuply ethoxylated glyceryl triacrylate (purity about 85% by weight). The amount used was 1.2 kg per t of monomer solution.

Free-radical polymerization is initiated using, per t of monomer solution, 1.39 kg of a 0.25% by weight aqueous hydrogen peroxide solution, 3.58 kg of a 15% by weight aqueous sodium peroxodisulfate solution and 1.28 kg of a 1% by weight aqueous ascorbic acid solution.

The monomer solution is introduced into a List Contikneter continuous kneader reactor with a capacity of 6.3 m³ (LIST AG, Arisdorf, Switzerland). The throughput of the monomer solution is about 20 t/h. The reaction solution has a feed temperature of 23.5° C.

Between the addition point for the crosslinker and the addition sites for the hydrogen peroxide and sodium peroxodisulfate solutions, the monomer solution is inertized with 4 m³/h of nitrogen. The monomer solution is metered into the reactor without separating off the nitrogen. The ascorbic acid solution is metered directly into the reactor in parallel with the monomer solution.

After about 50% of the dwell time, an additional about 1000 kg/h of polymer particles obtained in the production process by comminution and classification, having a particle size of less than 150 μm, is metered into the reactor. The dwell time of the reaction mixture in the reactor is about 15 minutes.

The polymer gel obtained is applied to the conveyor belt of an air circulation belt drier by means of an oscillating conveyor belt. The air circulation belt drier has a length of 48 m. The conveyor belt of the air circulation belt drier has an effective width of 4.4 m. On the air circulation belt drier, an air/gas mixture (about 175° C.) flows continuously around the aqueous polymer gel and dries it. The dwell time in the air circulation belt drier is 37 minutes.

The dried polymer gel is comminuted by means of a two-stage roll mill and sieved off to a particle size of 150 to 850 μm. Polymer particles having a particle size of less than 150 μm are separated off. Polymer particles having a particle size of greater than 850 μm are recycled into the comminution. Polymer particles having a particle size in the range from 150 to 850 μm are thermally surface postcrosslinked.

The polymer particles are coated with a surface postcrosslinker solution in a Schugi Flexomix® (Hosokawa Micron B.V., Doetinchem, the Netherlands) and then dried in a NARA Paddle Dryer (GMF Gouda, Waddinxveen, the Netherlands) at 185° C. for 45 minutes.

The following amounts are metered into the Schugi Flexomix®:

| | |
|---|---|
| 7.5 t/h | of polymer particles |
| 348.75 kg/h | of surface postcrosslinker solution |

The surface postcrosslinker solution comprises 2.2% by weight of 2-hydroxyethyl-2-oxazolidone, 2.2% by weight of propane-1,3-diol, 29.0% by weight of propane-1,2-diol, 3.2% by weight of aluminum sulfate, 56.9% by weight of water and 6.5% by weight of isopropanol.

After drying, the surface postcrosslinked polymer particles are cooled down to about 60° C. in a NARA Paddle-Cooler (GMF Gouda, Waddinxveen, the Netherlands).

The offgas is fed to a scrubbing column. The offgas consists essentially of the offgas from the drying and the offgas from the polymerization. The scrubbing column has a diameter of 5.9 m and a height of 19 m. The scrubbing column comprises random packings. At the top of the scrubbing column, about 530 m³/h of aqueous solution is applied. The aqueous solution consists of the bottoms liquid from the scrubbing column, about 4.7 m³/h of water and about 50 kg/h of aqueous sodium hydroxide solution. The pH of the aqueous solution is 10.5 to 11.0. The temperature of the aqueous solution is 60° C. The rest of the aqueous solution is discharged and used for production of the monomer solution. The content of acrylic acid and of neutralized acrylic acid in the aqueous solution is determined by NIR. The amounts of water, 50% by weight sodium hydroxide solution and acrylic acid in the neutralization are adjusted correspondingly.

pH values of the aqueous solution that are too low lead to inadequate separation of acrylic acid from the offgas stream. pH values of the aqueous solution that are too high lead to poorly reproducible discoloration and polymer gel in the recycled aqueous solution.

1. A process for continuously producing superabsorbents, comprising:
polymerizing an aqueous monomer solution comprising a partly neutralized acrylic acid to a polymer gel;
optionally extruding the polymer gel;
drying the polymer gel; and
comminuting, classifying, and optionally thermally surface postcrosslinking the dried polymer gel;
wherein:
a water content of the aqueous monomer solution is 40% to 75% by weight;
the acrylic acid has been neutralized to an extent of 40 to 85 mol %;
acrylic acid present in an offgas from the polymerization and/or drying is scrubbed out with an aqueous solution;
a pH of the aqueous solution is from 9.0 to 12.5; and
the acrylic acid-laden aqueous solution is used at least partly for production of the monomer solution or metered into the polymerization in parallel with the monomer solution.

2. The process according to claim 1, wherein the pH of the aqueous solution is 9.5 to 12.0.

3. The process according to claim 1, wherein the pH of the aqueous solution is 10.0 to 11.5.

4. The process according to claim 1, wherein the pH of the aqueous solution is 10.5 to 11.0.

5. The process according to claim 1, wherein the pH of the aqueous solution is adjusted with sodium hydroxide solution.

6. The process according to claim 1, wherein the aqueous solution is at a temperature of 50 to 70° C.

7. The process according to claim 1, wherein the acrylic acid is scrubbed out of the offgas with a scrubbing column.

8. The process according to claim 7, wherein a gas velocity in the scrubbing column is 1.0 to 2.0 m/s.

9. The process according to claim 7, wherein random packings are used in the scrubbing column.

10. The process according to claim 7, wherein a liquid hourly space velocity in the scrubbing column is 10 to 30 m³/h per m² of internal cross-sectional area of the scrubbing column.

11. The process according to claim 7, wherein the aqueous solution is partly circulated in the scrubbing column.

12. The process according to claim 11, wherein 97% to 99.7% of the aqueous solution is partly circulated in the scrubbing column.

13. The process according to claim 11, wherein the aqueous solution discharged from the scrubbing column comprises less than 97% by weight of water.

14. The process according to claim 1, wherein a content of acrylic acid and of neutralized acrylic acid in the monomer solution is determined by online analysis and amounts of acrylic acid, water and/or neutralizing agent used for the monomer solution are adjusted accordingly.
